(19) United States
(12) Patent Application Publication
(10) Pub. No.: US 2013/0056008 A1
(43) Pub. Date: Mar. 7, 2013

(54) PROTECTION DEVICES AND METHODS

(71) Applicants: GEORGE BONE, Silver Spring, MD (US); Ronald Jones, Bowie, MD (US)

(72) Inventors: GEORGE BONE, Silver Spring, MD (US); Ronald Jones, Bowie, MD (US)

(73) Assignee: LECARE, LLC, Largo, MD (US)

(21) Appl. No.: 13/569,420

(22) Filed: Aug. 8, 2012

Related U.S. Application Data
(60) Provisional application No. 61/521,167, filed on Aug. 8, 2011.

(51) Int. Cl. A61F 13/56; A61F 13/58; A61F 13/62; A61F 6/04; A61F 13/64

(52) U.S. Cl. 128/844; 604/385.03

(57) ABSTRACT

A protection device can include a first, barrier layer of moisture-proof material and a second, absorbing layer of absorbent material with respective openings therein for receiving a penis of a sexual partner during intercourse. The barrier layer is provided adjacent to the male sexual partner, and the absorbing layer material is provided adjacent to the female sexual partner. The absorbing material may absorb bodily fluids from intercourse while the barrier layer prevents such bodily fluids from reaching the male sexual partner, for example, to protect clothing or prevent bodily contact. The barrier and absorbing layers may have the same size in plan view. Alternatively, the barrier layer may be larger than the absorbing layer to provide an increased region of protection from bodily fluids. The protection device may also be provided with a condom, separately or coupled to the barrier and/or absorbing layers, to enhance protection from bodily fluids.

### CROSS-REFERENCE TO RELATED APPLICATIONS

The present application claims the benefit of U.S. Application No. 61/521,167, filed Aug. 8, 2011, which is incorporated by reference herein in its entirety.

### FIELD

The present application relates generally to devices for use during sexual intercourse, and, more particularly, to devices and methods for protection from bodily fluids during sexual intercourse.

### BACKGROUND

There are times when physical intimacy, such as sexual intercourse, between persons is desired, and yet one or more of the persons may be experiencing “spotting” or other undesirable bodily discharges. Depending upon the individuals, the circumstances may be inconvenient or too embarrassing to participate at that time. Consequently, the desires for intimacy go unsatisfied.

Also, there are times when physical intimacy, such as sexual intercourse (“sex”), between persons is desired and it may not be convenient or appropriate to remove one or more of the persons' clothing (e.g., “quickies”), which would probably result in soiled or stained clothing. Clothing removal may also be inappropriate when, for example, sex is desired but no skin contact between sexual partners is permissible for legal, religious or disease prevention reasons. Innumerable examples of such situations exist. Although finding privacy during “the big moment” may not be an issue, the results shown on the persons' clothing and the need for proper cleanup, would probably tell the story. Accordingly, there exists a need for devices and methods to protect one's person from bodily fluids associated with physical intimacy, such as sexual intercourse.

### SUMMARY

A protection device can include a first, barrier layer of moisture-proof material and a second, absorbing layer of absorbent material with respective openings therein for receiving a penis of a sexual partner during intercourse. The barrier layer is provided adjacent to the male sexual partner, and the absorbing layer material is provided adjacent to the female sexual partner. The absorbing material may absorb bodily fluids from intercourse while the barrier layer prevents such bodily fluids from reaching the male sexual partner, for example, to protect clothing or prevent bodily contact. The barrier and absorbing layers may have the same size in plan view. Alternatively, the barrier layer may be larger than the absorbing layer to provide an increased region of protection from bodily fluids. The protection device may also be provided with a condom, separately or coupled to the barrier and/or absorbing layers, to enhance protection from bodily fluids.

In embodiments, a protection device for use by a sexual partner during intercourse can include first and second layers. The first layer can have a first opening therein. The first layer can be formed of a flexible, moisture-proof material. The second layer can be disposed over the first layer and can be attached thereto. The second layer can have a second opening therein. A border of the second opening can enclose the first opening in plan view. The second layer can be formed of an absorbent material. The first layer can be constructed such that the first opening stretches to accommodate a penis inserted therein and to secure the protection device to said penis.

In embodiments, a protection method for use during sexual intercourse can include inserting a penis through a first opening in a barrier layer of a protection device. The barrier layer can be formed of a flexible, moisture-proof material such that the first opening stretches to accommodate the inserted penis and to secure the protection device thereto. The protection device can also include an absorbing layer disposed over the barrier layer and attached thereto. The absorbing layer can have a second opening therein. A border of the second opening can enclose the first opening in plan view. The absorbing layer can be formed of an absorbent material. The barrier layer can be disposed adjacent to a male sexual partner while the absorbing layer can be disposed adjacent to a female sexual partner during sexual intercourse, thereby protecting portions of the male sexual partner from bodily fluids.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described with reference to the accompanying drawings. Unless explicitly noted, the figures have not been drawn to scale. Where applicable, some features have not been illustrated to assist in the illustration and description of underlying features. Throughout the figures, like reference numerals denote like elements.

FIGS. 1A-1C show top, cross-sectional, and bottom views of a protection device, according to one or more embodiments of the disclosed subject matter.

FIG. 1D shows a cross-sectional view of a protection device with condom, according to one or more embodiments of the disclosed subject matter.

FIG. 1E shows a cross-sectional view of a protection device with a multi-layer condom, according to one or more embodiments of the disclosed subject matter.

FIG. 1F shows a top view of a protection device with extended barrier layer, according to one or more embodiments of the disclosed subject matter.

FIG. 2 shows a partially exploded perspective view of a protection device, according to one or more embodiments of the disclosed subject matter.

FIG. 3 shows a partially exploded perspective view of a protection device with condom, according to one or more embodiments of the disclosed subject matter.

FIG. 4 shows a close-up cross-sectional view of a multi-layer condom, according to one or more embodiments of the disclosed subject matter.

FIG. 5 shows a top view of a protection device with securing mechanisms, according to one or more embodiments of the disclosed subject matter.

FIG. 6 shows a front view of an undergarment incorporating a protection device, according to one or more embodiments of the disclosed subject matter.

FIG. 7 shows a cross-sectional view of a protection device with a condom and external anchor portion, according to one or more embodiments of the disclosed subject matter.

FIGS. 8A-8B show cross-sectional views of a protection device with a condom and internal anchor portion in initial and expanded states, respectively, according to one or more embodiments of the disclosed subject matter.

### DETAILED DESCRIPTION

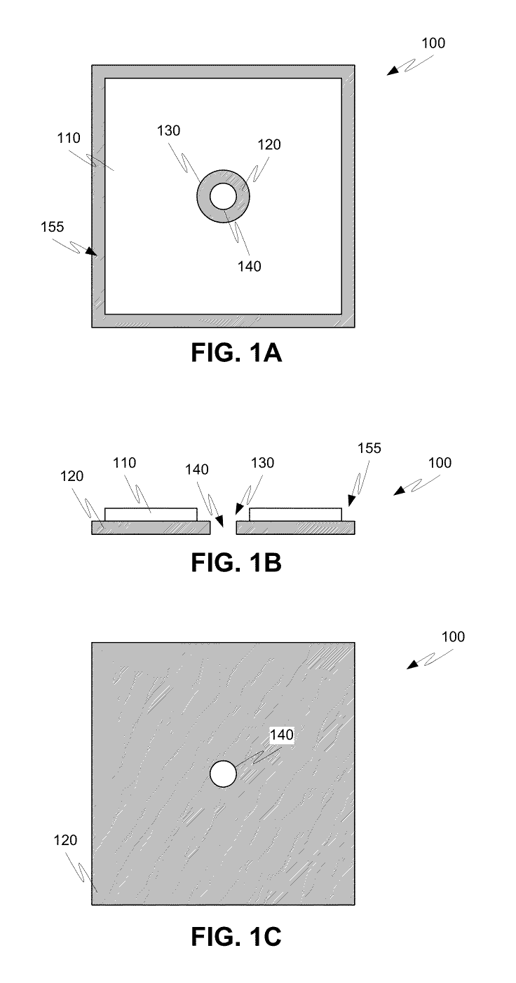

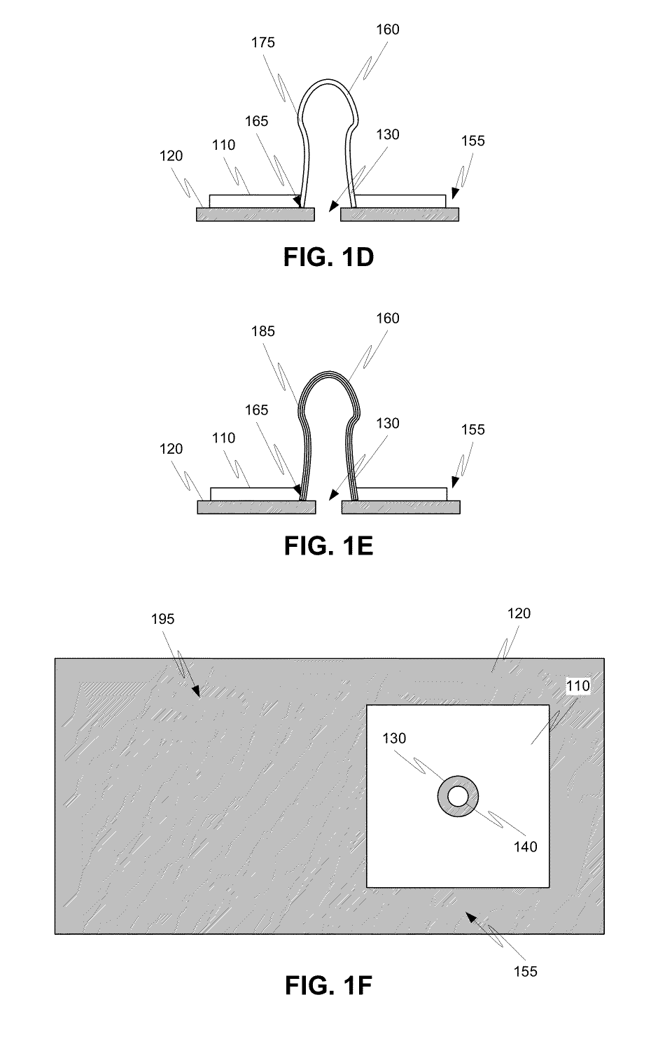

As shown in FIGS. 1A-1C, a protection device can include at least two layers of materials. A first layer 110 can be formed of an absorbent material and is thus referred to herein as an absorbing layer. A second layer 120 can be formed of a substantially fluid impervious material (i.e., moisture-proof) and is thus referred to herein as a barrier layer. Each of the first and second layers can have respective openings therein, through which a penis of the sexual partner is inserted for intercourse. In particular, the second layer 120 has an opening 140, which may be circular, oval, or any other shape. The first layer 110 has an opening 130, which may also be circular, oval, or any other shape. In general, the first layer opening 130 can be larger in size than the second layer opening 120, such that the border of the first layer opening 130 encloses the second layer opening 120, as shown in FIG. 1A. The second layer 120 is made of a sufficiently flexible and elastic material such that the opening 120 expands to accommodate insertion of the penis and to secure the protection device at the base of the penis after full insertion.

The protection device is arranged such that during sexual intercourse the absorbing layer 110 is adjacent to the female sexual partner and the barrier layer 120 is adjacent to the male sexual partner. Thus, bodily fluids produced by or incidental to sexual intercourse are absorbed by the absorbing layer 110 or at least prevented from reaching the male sexual partner by virtue of the barrier layer 120. Accordingly, the barrier layer 120 may have an extended size/shape to provide a larger area 195 of protection, as shown in FIG. 1F. Alternatively, the barrier layer 120 and the absorbing layer 110 may be substantially identical in size/shape, or the barrier layer 120 may have portions 155 that extend slightly beyond the absorbing layer 110 in plan view, as shown in FIGS. 1A-1C. The barrier layer 120 and/or the absorbing layer 110 can be configured as substantially planar layers coupled to each other, for example, along portions of facing surfaces thereof.

As an example of the functionality of the protection device, a physical intimacy scenario with a sexual position of a woman sitting on top of a man will be used. The man will sit or lie on his back and place the protection device in his lap with the absorbent material side up. The penis is inserted up through the two holes with the flexible material expanding to fit comfortably snug around the penis and pushed down to the base of the penis to prevent moisture from passing the flexible material. If a version of the protection device that does not include a condom is used, and a condom is desired, it can be added at this point. Either way, the woman can now sit on top of the man and begin sexual activities. During the sexual activity, the absorbent material can absorb all of the moisture. At the conclusion of the activity, the protection device is lifted off of the penis, folded over and placed back into a disposable pouch for proper disposal.

If safeguarding against HIV and other diseases that are transmitted via body fluids is a major concern during sexual activities, then a version of protection device that includes a condom may be utilized. This configuration can completely isolate the penis area from contact with any fluids or moisture. Considering the slippage and movement of regular condoms during sexual acts, using a condom by itself or adding a separate condom to the protection device may not provide as much protection against disease, moisture and body fluids as that provided by a version of the protection device that includes a permanently attached condom. Some of the problems related to the conventional condom during sexual activities have been skin irritation, leakage as it begins to slip, breakage as pressure is applied to the front end and/or having it slip completely off, thereby exposing the parties to various body fluids which could lead to disease and/or pregnancy.

The protection device 100 can be provided with a condom 160, for example, as shown in FIGS. 1D-1E. The condom 160 may be provided as a separate component independent from the barrier and absorbing layers of the protection device 100. Alternatively, the condom may be attached to the barrier and/or absorbing layers, for example, with an opening of the condom adjacent and/or sealed to a periphery of one or both of the openings in the barrier and absorbing layers. In another alternative, the condom may be integrally formed with the barrier layer, such that the condom and barrier layer form a unitary component. The opening of the condom 160 could thus serve as opening 140 of the barrier layer. Other configurations and combinations of one or more layers of the condom with one or more layers of the protection device 100 are also possible according to one or more contemplated embodiments.

The condom 160 may be placed over the penis prior to or after insertion of the penis through the openings of the protection device 100. Alternatively, the condom 160 can be attached to one of the barrier and absorbing layers of the protection device 100, for example, at a region 165 of the barrier layer 120 peripheral to opening 140, as shown in FIG. 1D. Inserting the penis into the openings of the protection device 100 thus allows simultaneous application of the condom 160 to the penis. Such a configuration may allow the protection device 100 to be used as a female condom, i.e., by placing condom 160 within the vagina prior to insertion of the penis into the protection device 100.

The condom 160 may comprise a single layer of material, for example, latex. Alternatively, the condom 160 may comprise multiple layers 185 having different properties, as shown in FIG. 1E. For example, the protection device of FIG. 1E can have a double-layer construction (DLC) condom. A lubricant (or any other substance) can be provided between inner and outer layers of the condom 160, with the two layers sealed at the base of the condom to barrier layer 120. This double-layer configuration can transfer much of the irritating friction-causing movement between the conventional condom surface and the skin of the participants from the outer surface of the conventional condom to the two inner lubricated surfaces of the double-layer configuration of the DLC condom. This configuration can provide a means for greater pleasure with significantly reduced friction, greater (i.e., double-layer) strength to guard against breakage, and can provide greater stability (i.e., reduced friction) to guard against slippage.

For example, an outer layer of the condom may be formed from a substantially hydrophobic material (e.g., a moisture-repelling yarn such as Lycra or Spandex) while the inner layer of the condom may be formed from a substantially hydrophilic material (e.g., a moisture-attracting yarn such as a soft molded non-latex polymer). Portions of the condom 160 and/or barrier layer 120 can be provided with stretch bands (e.g., by welding) to assist in keeping the protection device in place during intercourse. For example, portions 175 of condom 160 in FIG. 1D can be provided with stretch bands.

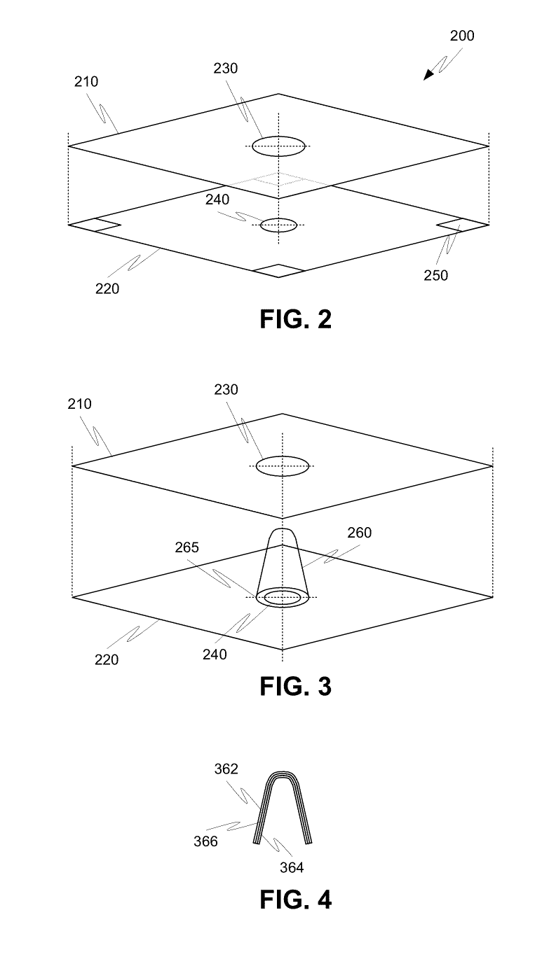

Referring to FIG. 2, an exemplary embodiment of a protection device 200 includes a sheet having two layers. A first layer 210 is formed as an area of an absorbent material, and a second layer 220 is formed as an area of a moisture-proof, flexible material such as a plastic. The first and second layers may be attached to each other along the periphery of the layers, or across the entire area of the layers, and may be attached to each other using any suitable means such as gluing, heat bonding, and taping. The first and second layers may be formed in various shapes including a square, rectangle, circle, and oval. The sheet may come in multiple sizes, ranging for example, from a square of 12 inches by 12 inches to a rectangle of 48 inches by 84 inches.

First layer 210 has a hole 230 going through the center of the first layer. Hole 230 may be, for example, 1.5 inches in diameter. Second layer 220 has a hole 240 going through the center of the second layer. Hole 240 is substantially smaller than hole 230 and may be, for example, 0.5 inches in diameter. Hole 230 is designed to comfortably expand or stretch to fit snugly around the perimeter of a penis of a person using protection device 200. Securing members 150 such as ties (not shown) or squares of a hook-and-loop material (e.g., Velcro®) may be included around the perimeter of second layer 220 to secure the protection device to a user's clothing and thus prevent movement of the protection device during physical intimacy such as sexual intercourse.

A separate condom (not shown) may be used in conjunction with protection device 200, to offer additional protection against contact with bodily fluids. Alternatively, a condom 260 can be permanently attached to the periphery of second hole 240 in second layer 220 and impermeably sealed over second hole 240, as shown in FIG. 3. Condom 260 may be integrally formed with second layer 220 as a unitary member or, alternatively, may be permanently attached to second layer 220 using any suitable means such as bonding, heat welding, or other methods used to manufacture condoms.

As noted above, the condom can be a single layer or multiple layers. For example, as shown in FIG. 4, the condom can include a first outer layer 362 and a second inner layer 364. The first outer layer 362 and the second outer layer 364 can be sealed at a base of the condom. A lubricant 366 can be disposed between the first outer layer 362 and the second outer layer 364. Condom 360 may be integrally formed with second layer 220 as a unitary member or, alternatively, may be permanently attached to either the first layer 210 or the second layer 220 using any suitable means such as bonding, heat welding, or other methods used to manufacture condoms.

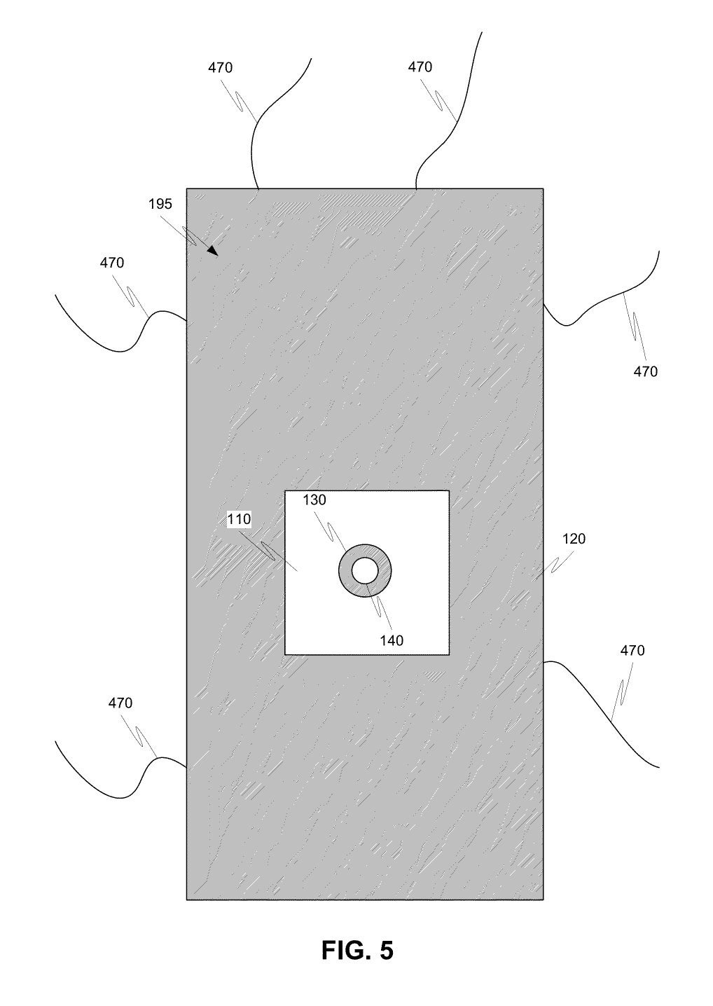

There are some religion-based and/or ethnicity-based sexual activities that require a man to only contact a woman with his penis during a specific sexual encounter and not have any other body contact. To accommodate the various religious and ethnic requirements related to these types of sexual activities, the protection device such as that shown in FIG. 5 is constructed such that the moisture-proof flexible backing (such as plastic) with the attached disposable absorbent material can be formed in various sizes larger than a 12-inch by 12-inch size. These sizes can vary from 12 inches by 12 inches, up to a full 48 inches by 84 inches. Regardless of the varying size of the flexible backing material, the 12-inch by 12-inch size of the area where the attached disposable absorbent material can still be maintained, since this is the only area that needs to absorb moisture and body fluids. Strategically located ties can be provided on the larger size protection devices to maintain the position of the protection device during sexual activity or other physical intimacy.

Thus, it may desirable to increase the size of the barrier layer 120 to increase the area protected, while the absorbing layer 110 may be maintained at a constant maximum size (e.g., 12 inches by 12 inches). For example, the barrier layer 120 may have planar dimensions from 12 inches by 12 inches up to 48 inches by 84 inches. In such configurations (or in other configurations disclosed herein), one or more securing mechanisms can be provided to hold the protection device in place during intercourse. For example, the securing mechanism can include one or more ties 470 arranged around the perimeter of the barrier layer 120. Ties 470 may serve to secure the protection device to a user's clothing. Alternatively or additionally, the securing mechanism can include one or more of hook-and-loop fasteners, straps, clips, ties, magnetic fasteners, buttons, and adhesive fasteners.

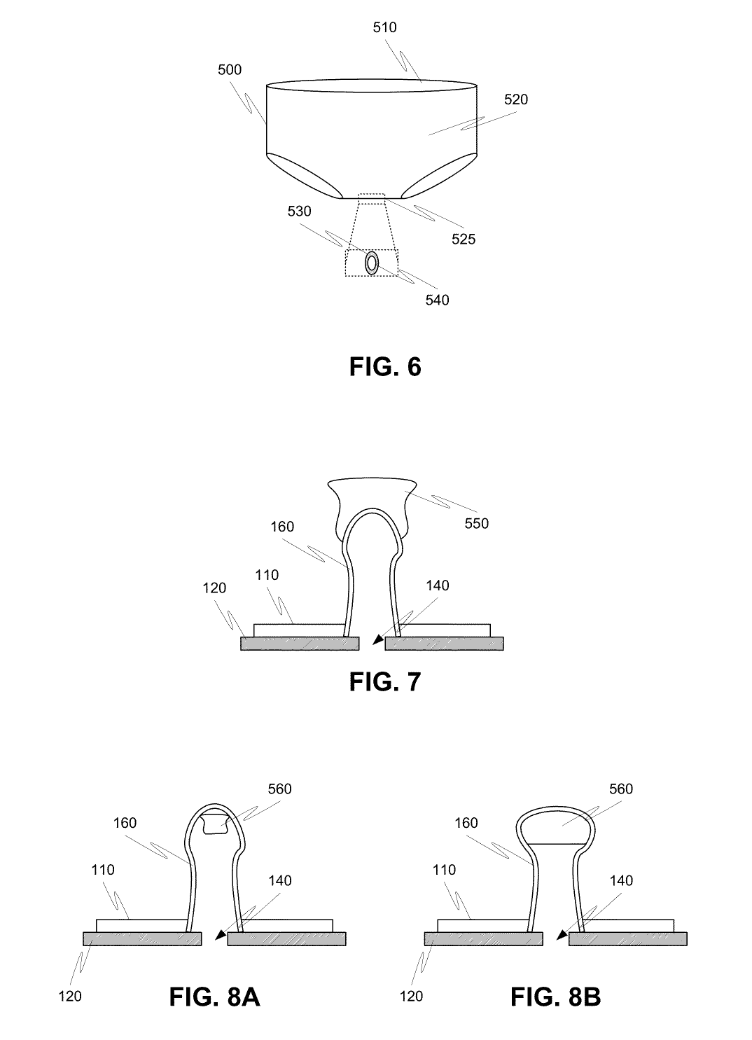

There may be times when sexual activity is spontaneous and/or it is not convenient or appropriate to carry a protection device around or pull a protection device out of a drawer or purse. To accommodate this situation, a version of the protection device such as that shown in FIG. 6 is provided in a configuration that a man or woman can wear the protection device like, for example, men's underwear (e.g., a brief, boxer or boxer brief) or women's underwear (e.g., a brief, bikini, thong or G-string panties). The protection device includes a moisture-proof flexible material such as plastic, shaped in the format of panties or underwear and is fully lined with disposable absorbent material. The absorbent material includes a relatively large, oblong hole in the front, center crotch area (e.g., approximately 1½″ long) and the flexible backing has a relatively small hole in the front, center crotch area (approximately ½″ in diameter), which can comfortably expand/stretch to the size of a user's penis.

A man can, for example, wear this protection device with the moisture-proof flexible material facing in against his skin and the absorbent material facing out. This version can also be turned inside out and worn by a woman with the absorbent material facing in against the woman's skin and the moisture-proof flexible material facing out. This version of the protection device can be utilized with a separate condom in a manner similar to that described above.

The protection device can be incorporated into an undergarment, such as underwear, as shown in FIG. 6. For example, a flexible undergarment 500 can have an inner layer 510 and an outer layer 520. When the undergarment is worn by a female sexual partner, the inner layer 510 can be formed of an absorbent material and the outer layer 520 can be formed of a moisture-proof material. Conversely, when the undergarment is worn by a male sexual partner, the inner layer 510 can be formed of a moisture-proof material and the outer layer 520 can be formed of an absorbent material. Respective openings of the layers of the undergarment 500 for insertion of the penis can be provided in a genital region 525 of the undergarment 500.

As shown in the enlarged inset of FIG. 6, the absorbing layer is provided with an opening 530, which may be oval in shape and the barrier layer is provided with an opening 540, which may also be oval. Although both openings are shown in the inset of FIG. 6, it is to be appreciated that only the opening 540 would be visible when the outer layer 520 is formed of the barrier material (i.e., moisture-proof material). Protection device 500 is constructed to be placed over a user's pelvis such that a penis can pass through the first and second holes. The undergarment can be constructed, for example, in the style of a brief, boxer, boxer-brief, panty, thong, and G-string. As with other disclosed embodiments, a separate condom or integrated condom may be used in conjunction with protection device 500 to offer additional protection against contact with bodily fluids.

For example, a version of the protection device such as that shown in FIG. 6 can include an integrated condom. This version may additionally include a double-layer construction (DLC) condom permanently attached and sealed over the hole of the flexible backing layer. This configuration can provide the DLC condom benefits discussed above. A man can also wear this protection device with the moisture-proof flexible material facing in against his skin and the absorbent material out against the woman's skin. This version can also be turned inside out and worn by the woman with the absorbent material facing in against the woman's skin and the moisture-proof flexible material out against the man's skin.

The protection device with integrated condom can be configured as a female condom, whereby the condom is inserted into the vagina of the sexual partner prior to insertion of the penis into the protection device. In such configurations, the initiation of intercourse may cause uncomfortable movement of the condom within the vagina. The condom may thus be provided with an anchor portion to secure an end of the condom distal from the layers of the protection device within the vagina prior to and during intercourse. For example, condom 160 can include an external anchor portion 550, as shown in FIG. 7. External anchor portion 550 can be, for example, a sponge or diaphragm. Alternatively or additionally, condom 160 can include an internal anchor portion 560, as shown in FIGS. 8A-8B. Internal anchor portion 560 can be, for example, a sponge that expands upon absorption of fluid. Thus, the sponge 560 can expand from an initial state, as shown in FIG. 8A, to an expanded state, as shown in FIG. 8B, to secure the condom within the vagina.

The absorbent material and/or the flexible material of the various versions of protection device can be formed in various colors including yellow, red, brown, dark brown or burgundy to minimize the contrast of the moisture collected and to reduce potential embarrassment to the users. The surfaces of the moisture-proof flexible material on the protection device can provide a place for a manufacturer's logo and/or advertisement, as well as other paid advertisements. The disposable resealing pouch (not shown) may also provide one or more surfaces that would be available for a manufacturer's logo and/or advertisement, as well as other paid advertisements. The protection device may also be packaged to include a condom with the versions that do not have a permanently attached condom (e.g., that shown in FIGS. 1A-1C, 1F, 2, 5, and 6). Additionally, the protection device may be packaged to include disposable wipes in the protection device disposable resealing pouch.

Additional potential applications for the devices and methods described herein include so called “lap dances,” sexual contact, including but not limited to oral sex, anal sex, and vaginal sex, as well as other forms of physical intimacy that may be spontaneous and may not readily support the removal of the participants' clothing. Protection of one's person, especially clothing being worn during the physical intimacy, may be afforded by the devices and methods described herein.

An exemplary protection device for physical intimacy may include a flexible sheet having a first layer and a second layer, the first layer being absorbent and having a first hole through the first layer, the second layer being impermeable and elastic and having a second hole through the second layer, the first and second holes being concentric, with a diameter of the second hole being smaller than a diameter of the first hole, where the device is constructed to be placed over a user's penis such that the penis passes through the second hole and then the first hole, and the perimeter of the second hole stretches to fit snugly around the user's penis. The sheet may be rectangular and can range in size from 12 inches by 12 inches, up to 48 inches by 84 inches. Alternatively, the sheet may be square and measure 12 inches by 12 inches. The device may further include a plurality of tie members attached to the periphery of the sheet and constructed to maintain the position of the device on the user during the physical intimacy. Alternatively or additionally, the second layer may include securing members affixed to the periphery of the second layer and constructed to hold the device firmly in place against a user's clothing. The securing members can be affixed to corners of the second layer. The diameter of the first hole may be approximately 1.5 inches, and the diameter of the second hole may be approximately 0.5 inches. The device may further include a disposable pouch constructed to store the sheet prior to its use and to facilitate disposal of the sheet after its use. The device may further include a condom permanently attached to the periphery of the second hole in the second layer and impermeably sealed over the second hole. The condom may include a first outer layer and a second inner layer, the first and second layers being sealed at a base of the condom, and a lubricant disposed between the first and second layers. The first and second layers of the sheet may be formed in one of a yellow, red, brown, and burgundy color, such that color contrast of fluid collected during the physical intimacy is minimized. The second layer of the sheet may be printed with one or more of a manufacturer logo, a manufacturer advertisement, and a paid advertisement.

An exemplary method for protecting a first person from bodily fluids of another person during physical intimacy may include: providing a protection device including a flexible sheet having a first absorbent layer and a second impermeable layer, the first layer having a first through hole, the second layer being elastic and having a second through hole, the first and second through holes being concentric with a diameter of the second through hole being smaller than a diameter of the first through hole; placing the protection device over the first person's penis such that the penis passes through the first and second through holes, the first layer faces away from the first person, and the perimeter of the second hole stretches to fit snugly around the user's penis; removing the protection device from the first person's penis following the physical intimacy; and disposing of the protection device. The disposing of the protection device may include placing the sheet into a disposable pouch. The method may further include placing a condom over the first person's penis prior to placing the protection device over the first person's penis. Alternatively, the method may include placing a condom over the first person's penis after placing the protection device over the first person's penis. The device may include a condom permanently attached to the periphery of the second hole in the second layer and impermeably sealed over the second hole. The condom may include a first outer layer and a second inner layer, the first and second layers being sealed at a base of the condom, and a lubricant disposed between the first and second layers.

An exemplary protection device for physical intimacy may include a flexible sheet having a first layer and a second layer, the first layer being absorbent and having a first hole through the first layer, the second layer being impermeable and elastic and having a second hole through the second layer, the first and second holes being concentric, with a diameter of the second hole being smaller than a diameter of the first hole, where the device is constructed to be placed over a user's penis such that the penis passes through the second hole and then the first hole, and the perimeter of the second hole stretches to fit snugly around the user's penis. The sheet may be rectangular and can range in size from 12 inches by 12 inches, up to 48 inches by 84 inches. Alternatively, the sheet may be square and measure 12 inches by 12 inches. The device may further include a plurality of tie members attached to the periphery of the sheet and constructed to maintain the position of the device on the user during the physical intimacy. Alternatively or additionally, the second layer may include securing members affixed to the periphery of the second layer and constructed to hold the device firmly in place against a user's clothing. The securing members can be affixed to corners of the second layer. The diameter of the first hole may be approximately 1.5 inches, and the diameter of the second hole may be approximately 0.5 inches. The device may further include a disposable pouch constructed to store the sheet prior to its use and to facilitate disposal of the sheet after its use. The device may further include a condom permanently attached to the periphery of the second hole in the second layer and impermeably sealed over the second hole. The condom may include a first outer layer and a second inner layer, the first and second layers being sealed at a base of the condom, and a lubricant disposed between the first and second layers. The first and second layers of the sheet may be formed in one of a yellow, red, brown, and burgundy color, such that color contrast of fluid collected during the physical intimacy is minimized. The second layer of the sheet may be printed with one or more of a manufacturer logo, a manufacturer advertisement, and a paid advertisement.

An exemplary method for protecting a first person from bodily fluids of another person during physical intimacy may include: providing a protection device including a flexible sheet having a first absorbent layer and a second impermeable layer, the first layer having a first through hole, the second layer being elastic and having a second through hole, the first and second through holes being concentric with a diameter of the second through hole being smaller than a diameter of the first through hole; placing the protection device over the first person's penis such that the penis passes through the first and second through holes, the first layer faces away from the first person, and the perimeter of the second hole stretches to fit snugly around the user's penis; removing the protection device from the first person's penis following the physical intimacy; and disposing of the protection device. The disposing of the protection device may include placing the sheet into a disposable pouch. The method may further include placing a condom over the first person's penis prior to placing the protection device over the first person's penis. Alternatively, the method may include placing a condom over the first person's penis after placing the protection device over the first person's penis. The device may include a condom permanently attached to the periphery of the second hole in the second layer and impermeably sealed over the second hole. The condom may include a first outer layer and a second inner layer, the first and second layers being sealed at a base of the condom, and a lubricant disposed between the first and second layers.

An exemplary protection device for physical intimacy may include a flexible undergarment including a first layer and a second layer, the first layer being absorbent and having a first hole through the first layer, the second layer being impermeable and elastic and having a second hole through the second layer, the first and second holes being concentric, with the second hole being smaller than the first hole, where the device is constructed to be placed over a user's pelvis such that a penis can pass through the first and second holes. The first and second holes may be oblong in shape. The first hole may be approximately 1.5 inches in length. The second hole may be approximately 0.5 inches in length. The undergarment may be constructed in the style of a brief, boxer, boxer-brief, panty, thong, or G-string. The device may also include a condom permanently attached to the periphery of the second hole in the second layer and impermeably sealed over the second hole. The condom may include a first outer layer and a second inner layer, the first and second layers being sealed at a base of the condom, and a lubricant disposed between the first and second layers. The first and second layers of the sheet may be formed in one of a yellow, red, brown, and burgundy color, such that color contrast of fluid collected during the physical intimacy is minimized. The second layer of the sheet may be printed with one or more of a manufacturer logo, a manufacturer advertisement, and a paid advertisement.

An exemplary method for protecting a first person from bodily fluids of another person during physical intimacy may include: providing a protection device including a flexible undergarment having a first layer and a second layer, the first layer being absorbent and having a first through hole, the second layer being impermeable and elastic and having a second through hole, the first and second through holes being concentric, with a diameter of the second through hole being smaller than a diameter of the first through hole; placing the protection device over the first person's pelvis such that a penis can pass through the first and second through holes; removing the protection device from the first person's pelvis following the physical intimacy; and disposing of the protection device. The placing the protection device over the first person's pelvis may include configuring the undergarment with the first layer on the interior, such that the absorbent first layer contacts the first person's pelvis. Alternatively, the placing the protection device over the first person's pelvis may include configuring the undergarment with the first layer on the exterior, such that the impermeable second layer contacts the first person's pelvis. The perimeter of the second hole can stretch to fit snugly around a penis of the first person. The method may include placing a condom over the penis prior to placing the protection device over the first person's pelvis. Alternatively, the method may include placing a condom over the penis after placing the protection device over the first person's pelvis. The disposing of the protection device may include placing the undergarment into a disposable pouch.

Features of the disclosed embodiments may be combined, rearranged, omitted, etc., within the scope of the invention to produce additional embodiments. Furthermore, certain features may sometimes be used to advantage without a corresponding use of other features.

It is, thus, apparent that there is provided, in accordance with the present disclosure, protection devices and associated methods. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicants intend to embrace all such alternatives, modifications, equivalents, and variations that are within the spirit and scope of the present invention.

1. A protection device for use by a sexual partner during intercourse, the protection device comprising:
a first layer having a first opening therein and being formed of a flexible, moisture-proof material;
a second layer disposed over the first layer and attached thereto, the second layer having a second opening therein, a border of the second opening enclosing the first opening in plan view, the second layer being formed of an absorbent material,
wherein the first layer is constructed such that the first opening stretches to accommodate a penis inserted therein and to secure the protection device to said penis.

2. The protection device of claim 1, wherein the first layer includes a portion that extends beyond at least one edge of the second layer in plan view, the extended portion being constructed to protect a body of the sexual partner from bodily fluids during intercourse.

3. The protection device of claim 1, wherein the first and second layers are substantially planar.

4. The protection device of claim 1, further comprising a condom disposed such that the penis inserted into the first opening is enclosed by said condom.

5. The protection device of claim 4, wherein said condom is attached to the first layer at a region of the first layer bordering said first opening.

6. The protection device of claim 4, wherein said condom includes at least an inner layer, an outer layer, and a lubrication layer disposed between the inner and outer layers.

7. The protection device of claim 6, wherein the inner layer includes a hydrophilic material and the outer layer includes a hydrophobic material.

8. The protection device of claim 6, wherein the inner layer includes a soft-molded non-latex polymer, and the outer layer includes a stretchable synthetic fiber.

9. The protection device of claim 1, wherein the first layer has a minimum dimension in plan view of at least 12 inches.

10. The protection device of claim 1, wherein the first and second layers form a portion of an undergarment for the sexual partner.

11. The protection device of claim 1, further comprising at least one securing mechanism coupled to at least one of the first and second layers, each securing mechanism being constructed to releasably attach the protection device to the sexual partner.

12. The protection device of claim 1, wherein the at least one securing mechanism includes one of a hook-and-loop fastener, a strap, a clip, a tie, and an adhesive fastener.

13. The protection device of claim 1, wherein the first and second openings are oval or circular, a maximum planar dimension of the second opening is approximately 1.5 inches, and a maximum planar dimension of the first opening is approximately 0.5 inches.

14. The protection device of claim 1, further comprising a condom and an anchor portion, wherein the condom is disposed such that the penis inserted into the first opening is enclosed by said condom, the anchor portion is provided at an end of the condom distal from the first and second layers, and the anchor portion is constructed to retain the condom in a vagina of a sexual partner before or during the intercourse.

15. A protection method for use during sexual intercourse, the method comprising:
inserting a penis through a first opening in a barrier layer of a protection device, the barrier layer being formed of a flexible, moisture-proof material such that the first opening stretches to accommodate the inserted penis and to secure the protection device thereto,
wherein the protection device further comprises an absorbing layer disposed over the barrier layer and attached thereto, the absorbing layer having a second opening therein, a border of the second opening enclosing the first opening in plan view, the absorbing layer being formed of an absorbent material, and
the barrier layer is disposed adjacent to a male sexual partner while the absorbing layer is disposed adjacent to a female sexual partner during said sexual intercourse, thereby protecting portions of the male sexual partner from bodily fluids.

16. The method of claim 15, wherein the inserting a penis occurs prior to the sexual intercourse.

17. The method of claim 15, wherein the protection device is provided on the female sexual partner and the inserting occurs simultaneously with the initiation of sexual intercourse.

18. The method of claim 15, wherein a condom is attached to the protection device such that the inserting simultaneously places the condom on said penis.

19. The method of claim 15, further comprising, placing a condom on said penis independently of said inserting.

20. The method of claim 15, further comprising, after the sexual intercourse, removing the protection device from the penis, and disposing of the protection device.
